(19) United States
(12) Patent Application Publication
(10) Pub. No.: US 2011/0098495 A1
(43) Pub. Date: Apr. 28, 2011

(54) Compositions and methods of omega polyunsaturated fatty acids for the treatment of oral diseases

(75) Inventor: Chifu Huang, Lexington, KY (US)

(73) Assignee: Chifu Huang, Lexington, KY (US)

(21) Appl. No.: 12/606,257

(22) Filed: Oct. 27, 2009

(51) Int. Cl. C07C 57/03

(52) U.S. Cl. 554/224

(57) ABSTRACT

The invention discloses a novel discovery of anti-microbial activity against oral bacteria, which can be applied for controlling and preventing oral diseases, of omega-3, omega-6, and omega-9 fatty acids, but not limited to omega-3, omega-6, and omega-9 fatty acids; omega-3, omega-6, and omega-9 fatty acid methyl esters, and omega-3, omega-6, omega-9 fatty acid ethyl esters and their application method. The application of this discovery includes the preparation of drugs, dietary supplements, food and daily necessities with oral health care and therapeutic effects.

### FIELD OF THE INVENTION

The present invention relates to omega-3, omega-6, and omega-9 fatty acids, and their ester derivatives, and discovery of anti-microbial activity of omega-3, omega-6, and omega-9 fatty acids, their ester derivatives, and the novel use of omega-3, omega-6, and omega-9 fatty acids. Specifically, the omega-3 (EPA, DHA, ALA), omega-6 fatty acids (ARA, GLA), and omega-9 fatty acids, and their fatty acid methyl esters and ethyl esters found in fish oil, algae oil or plant oil have anti-microbial activity against oral pathogens, and the application in the prevention and treatment of oral diseases.

### BACKGROUND OF THE INVENTION

Omega-3 fatty acids include eicosapentaenoic acid (EPA), docosahexaenoic acid (DHA), α-linolenic acid (ALA) and others, belonging to n-3 unsaturated fatty acids (also known as the ω-3 unsaturated fatty acids or omega-3 fatty acid) family. Their common feature, the first double bond in the carbon chain located at third position, has played an important role in physiological functions. Although n-3 fatty acids are vital to good health and are considered one of the essential fatty acids, they can not be synthesized in the human body. Omega-6 fatty acids, such as arachidonic acid (ARA) and γ-linolenic acid (GLA), also play an important physiological function.

Over the past 20 years, scientists focus on the physiological and biochemical role of fish oil's EPA, DHA and ALA. Two main isomers of linolenic acid were found: α-linolenic acid (ALA) and γ-linolenic acid (GLA). GLA, an n-6 polyunsaturated fatty acid, is the precursor in synthesis of arachidonic acid (arachidonic acid, ARA); ALA is the n-3 polyunsaturated fatty acids. Currently, the main function of ALA is the precursor in synthesis of n-3 polyunsaturated fatty acids (EPA and DHA); Eicosapentaenoic acid (EPA), an important polyunsaturated fatty acid complex, and chemical messengers play a crucial role in the immune and inflammatory responses. Various n-3 and n-6 arachidic acids are linked in cell membrane, where the n-3 arachidic acid desaturases to EPA. In the inflammatory response, ALA and EPA transform to other n-20 derivatives via biological activity by cyclooxygenase and lipoxygenase. Docosahexaenoic acid (DHA) is required for retinal development and normal function. The study indicated that during the critical period of development, short supply of DHA will have permanent defects in retinal function. In addition, DHA plays a crucial role of the nervous system function because it can protect nerve cells (neurons) to avoid programmed cell death (from apoptosis), and enhance the viability of the nervous system. In general, EPA, DHA and ALA are important because they not only inhibit the promotion of prostaglandin and thromboxane formation (in order to protect vascular endothelial cells, inhibit platelet aggregation, prevent atherosclerotic plaque and vascular thrombosis), but are also essential to the fetus and the normal development of infants and young children.

Due to the human body's essential fatty acids found in fish oil, the fatty acids are widely used in functional food. However, no scientific research on omega-3, omega-6 fatty acids with anti-microbial activity against oral pathogens has been reported. No application of omega-3 and omega-6 fatty acids is available to utilize their anti-microbial activity against oral pathogens to treat oral diseases. Based on our research results of this invention (i.e. omega-3, omega-6, omega-9 fatty acids in fish oil have novel anti-microbial activity), a novel bioactivity of omega-3, omega-6, and omega-9 fatty acids is discovered and a novel application of the omega-3 and omega-6 fatty acids is developed. There is no prior use in anti-oral bacterial application.

As we all know, since the birth of antibiotic drugs, their power of eradicating bacteria have saved countless lives. However, the increased use of antibiotics for infection treatment has allowed for the greater possibility of genetic mutation in bacteria. Even more frightening is that once the drug-resistant bacteria appear, antibiotics will lose their effectiveness. At present, the excessive use of antibiotics is a global problem, which relates to medical, social and public health. Therefore, there is a strong need and market demand for anti-bacterial compounds or products which have low toxicity and high bioactivity against oral bacteria. There is a strong need and market for antibacterial compounds and products to treat oral bacterial and to improve oral health, which produce local not systemic treatment/effect.

The present invention, using the novel anti-microbial activity of omega-3, omega-6, and omega-9 fatty acids, their methyl esters and developed and applied in products for health care and as an alternative solution for oral infectious diseases. The current application will meet the market needs for anti-oral bacteria agents and has great commercial values.

The current invention utilizes the novel discovery of anti-microbial activity, which can be applied for controlling and preventing oral diseases, of omega-3 and omega-6 fatty acids, their acid methyl esters, and omega-3 fatty acid ethyl esters and their application method. The common omega-3 fatty acids include eicosapentaenoic acid (EPA), docosahexaenoic Acid (DHA), α-linolenic acid (ALA), omega-6 fatty acids (ARA, GLA).

The application by this discovery includes the preparation of drugs, dietary supplements, food and daily necessities with oral health care and therapeutic effects. The drugs with therapeutic effect can be in the form of tablet, spray, powder, film, paint, ointment, gel, liquid, foam, and rinse agent; while the food and daily necessities comprise chewing gum, drinks, tea, milk, sugar, tooth paste, dentifrice. Provided by the invention of omega-3, omega-6, and omega-9 fatty acids, their methyl esters, and ethyl esters, having anti-microbial activity for the prevention and treatment of oral diseases, is the first time that fish oil fatty acids, fish oil fatty acid methyl esters, and fish oil fatty acid ethyl esters were applied to oral diseases as anti-microbial agents against oral pathogens, such as Streptococcus mutans (ATCC 25175), Porphyromonas gingivalis ATCC 33277, A. actinomycecomitan JP2, F. nucleatum ATCC 25586, and yeast Candida albicans ATCC 2091; also the first time omega-3 and omega-6 fatty acids being used for prevention and treatment of dental caries, periodontitis, gingivitis, toothache and bleeding, mouth ulcers, halitosis, pharyngitis, sore throat and other oral diseases, drugs, health food and daily necessities. Application prospects have important social, economic and health benefits.

### SUMMARY OF THE INVENTION

The purpose of the present invention is to provide a novel application of omega-3, omega-6, and omega-9 fatty acids, using omega-3, omega-6, and omega-9 fatty acids, their methyl esters, and ethyl esters, to ensure the full exploitation of the anti-microbial activity for human health services. The characteristics are the discovery of anti-microbial activity of omega-3, omega-6, and omega-9 fatty acids against oral bacteria and their application for oral diseases. Specifically, the fatty acids, fatty acid methyl esters, and fatty acid ethyl esters found in fish oil or algae or plants, have significant anti-microbial activity, allowing them to be applied to the prevention and treatment of oral diseases, such as caries, periodontitis, and gingivitis. The application is a novel use of pre-existing compounds which are already proved safe. And no prior art of such use exists.

The invention will make the methods for the application of anti-microbial activity of common omega-3 fatty acids (EPA, DHA, and ALA) and omega-6 fatty acid (ARA), and omega-9, their fatty acid methyl esters, and fatty acid ethyl esters and their application of prevention of oral diseases. The characteristics of the above methods and compounds are:
(1) Common omega-3 fatty acids (EPA, DHA, ALA) and omega-6 fatty acid (ARA, GLA), and their fatty acid methyl esters, and fatty acid ethyl esters have anti-microbial activity; novel anti-microbial activity against oral pathogens. Through research, the inventor found that, the omega-3 fatty acids (EPA, DHA, ALA), omega-6 fatty acid (ARA, GLA), omega-3 and omega-6 fatty acid methyl esters, omega-3 and omega-6 fatty acid ethyl esters have strong anti-microbial bioactivity. The biological activity has a strong inhibitory effect on oral microorganisms, particularly Streptococcus mutans, Porphyromonas gingivalis, Candida albicans, Actinobacillus actinomycetemcomitans, Fusobacterium nucleatum, but not limited.

The inventor verified the test by dissolving 1 mg of omega-3 fatty acids (EPA, DHA, and ALA) and omega-6 fatty acids (ARA, GLA), omega-3 fatty acid methyl esters (EPA-ME, DHA-ME, ALA-ME), and omega-3 fatty acid ethyl esters (EPA-EE, DHA-EE, ALA-EE) in 1000 μl ethanol solution; took 1 (final concentration 1 μg/ml) and 5 μl (final concentration 5 μg/ml) solution by adding 100 μl of Streptococcus mutans in the culture medium cultured overnight at 37° C. After the overnight culture, the medium was diluted 1:10000 times, 5 μl of the diluted culture was applied to blood agar plate and cultured overnight. Calculated the number of colonies on the plates and compared with the control group to obtain the percentage of inhibition. It was found that the 1 ug/ml final concentration of omega-3 fatty acids, omega-3 fatty acid methyl esters, and omega-3 fatty acid ethyl esters could inhibit 50% of Streptococcus (Streptococcus mutans) growth; the final concentration of 5 μg/ml fatty acids could inhibit 50% of Porphyromonas gingivalis growth. The results showed that omega-3 fatty acids, omega-3 fatty acid methyl esters, and omega-3 fatty acid ethyl esters, have strong antibacterial activity. Among them, the strongest antibacterial activity was found in omega-3 fatty acids. Also omega-3 fatty acids (EPA, DHA, ALA) and omega-6 fatty acids ARA can inhibit 50% of Candida albicans at final concentration of 25 μg/ml; with Actinobacillus actinomycetemcomitans at a final concentration of 15 μg/ml.

(2) Fish oil/omega-3 and omega-6 fatty acids, fish oil fatty acid methyl ester, and fish oil fatty acid ethyl ester are to be used in prevention and treatment of oral diseases The present invention provides an application for oral disease prevention and control by omega-3 and omega-6 fatty acids, their fatty acid methyl esters and ethyl esters. The method for prevention and control of oral infection mentioned above is that omega-3, omega-6, and omega-9 fatty acids, their fatty acid methyl esters, and fatty acid ethyl esters are prepared for oral health care, 0.01-20% of the content to the final product weight, where the specific content items based on the type of system and requirements.

The above-mentioned oral diseases include dental caries, periodontitis, gingivitis, toothache and bleeding, mouth ulcers, halitosis, pharyngitis, sore throat and so on. As a result, omega-3 and omega-6 fatty acids, their fatty acid methyl esters and ethyl esters are biologically active components, having a strong oral anti-microbial role. The components will be used for the prevention and treatment of oral diseases and should receive good results.

Omega-3, omega-6, and omega-9 fatty acids, their methyl esters, ethyl esters are the extracts from fish oil, algae oil and plant oil, including the common fatty acids (EPA, DHA, ALA, ARA, GLA) and other unusual fatty acids in the sum.

Oral health care and therapeutic products include medicines, food, and daily necessities. Formulations of these products include the fish oil fatty acids, fish oil fatty acid methyl ester, fish oil fatty acid ethyl ester, additives and the appropriate form of the product carrier. In accordance with these products, they are prepared by conventional production methods.

The role of oral treatment with these drugs can be used in formulations such as tablets, sprays, powder, film, paint, ointment, gel, liquid, foam, mouthwash, etc. The treatments mainly refers to the tablets for oral medication in various form of tablets, including oral tablets, bio-adhesive patches, dispersible tablets, effervescent tablets, chewable tablets, tablets and other solutions. The fish oil fatty acids, fish oil fatty acid methyl ester, and fish oil fatty acid ethyl ester in the above-mentioned drugs should account for at least 0.1 percent to the weight of the mixture of drugs. The more effective composition would generally be 0.01-20%.

The key role as mentioned above includes functional food such as chewing gum, beverages, toffee, and other dairy products. The fish oil fatty acids, fish oil fatty acid methyl ester, and fish oil fatty acid ethyl ester content should be at least 0.01% to weight mixture of food, although 0.01-20% would be ideal.

The key role as mentioned above includes oral daily necessities such as toothpaste, tooth powder, etc. The fish oil fatty acids, fish oil fatty acid methyl ester, and fish oil fatty acid ethyl ester content should be at least 0.01% to the weight of a mixture of daily necessities, although 0.01-20% would be ideal.

Provided by the invention of omega-3 fatty acids, omega-6 fatty acids, their fatty acid methyl ester, and ethyl ester having anti-microbial activity for the prevention and treatment of oral diseases, is the first time that omega fatty acids were applied to prevention and treatment of oral diseases. The invention was primarily developed for the prevention and treatment of dental caries, periodontitis, gingivitis, toothache and bleeding, mouth ulcers, halitosis, pharyngitis, sore throat, other oral diseases, drugs, health food and daily necessities, but not limited to. Application prospects have important social and economic benefits.

### DETAILED DESCRIPTION OF THE INVENTION

The following examples are presented by way of illustration, not of limitation.

### Example 1

Omega-3 and omega-6 fatty acids (EPA, or DHA, or ALA, or ARA, or GLA) as raw materials are catalyzed to esters with following reaction method. Add omega-3 fatty acids in solution of anhydrous ethanol/HC1 molar ratio of reactants 10: at 75° C. ˜77° C., for 2 hours, with acid catalyst concentration about 0.8N˜1.0N. Afterwards, add hexane to the mixture to extract fatty acid ethyl esters and obtain (EPA-EE, or DHA-EE, or ALA-EE or ARA-EE). Or using methanol as solvent, fish oil fatty acid methyl ester (EPA-ME, DHA-ME, ALA-ME, ARA-ME) can also be carried out in a similar way.

### Example 2

Dissolve 1 mg omega-3 or omega-6 fatty acids (EPA, or DHA, or ALA or ARA or GLA) in 10 ml ethanol to make a 1 mg/ml stock solution. Take 1 μl and 5 μl of above solution and add into 100 μl Streptococcus mutans culture (at final concentration 1 μg/ml and 5 μg/ml), incubate the said bacterial culture overnight at 37° C. Afterwards, dilute the overnight culture by 1:10000. Five μl of the diluted culture mix was applied to blood agar plate and cultured overnight. Calculate the clone number on the plate and compared with the negative control plate. Final concentration of 1 μg/ml of omega-3 and omega-6 fatty acids could significantly inhibit the growth of Streptococcus mutans. The result shows that the IC₅₀ of omega-3 fatty acid is approximately 1 μg/ml. IC₅₀ of omega-6 fatty acids (ARA and GLA) is also similar to 5 μg/ml.

### Example 3

Dissolve 1 mg of omega-3 fatty acid ethyl ester (EPA-EE, or DHA-EE, or ALA-EE) in 10 ml ethanol. One μl and 5 μl of the above solution were added into 100 μl of Streptococcus mutans culture (at final concentration 1 μg/ml and 5 μg/ml), and cultured overnight at 37° C. Dilute this culture by 1:10000. Five μl of diluted solution was applied to blood agar plate and cultured overnight. Calculate the clone number on the disk, and compared with the negative control. Results showed that fish oil ethyl esters have significant anti-bacterial activity. Final concentration of 1 μg/ml extract could inhibit the growth of Streptococcus mutans. The result shows that fish oil fatty acid ethyl ester IC₅₀ is 2.5 μg/ml approximately.

### Example 4

Dissolve 1 mg of omega-3 fatty acid methyl ester (EPA-ME, or DHA-ME, or ALA-ME) in 10 ml ethanol. One μl and 5 μl were added into 100 μl Streptococcus mutans solution (at final concentration 1 μg/ml and 5 μg/ml), and then cultured overnight at 37° C. Dilute this culture by 1:10000 and apply 5 μl of diluted solution to blood agar plate, and cultured overnight. Calculate the clone number on the plate and compare with the negative control. Results showed that the omega-3 fatty acid methyl esters have significant anti-bacterial activity. Final concentration of 1 μg/ml omega-3 fatty acid methyl ester could inhibit the growth of Streptococcus mutans. The study showed that omega-3 fatty acid methyl esters have IC₅₀ of 2.5 μg/ml approximately.

### Example 5

Took 100 grams of algae powder, for example, chlorella powder and crushed in 500 ml ethanol/HC1 acid solution and boiled for 60 minutes. Extract will be filtered and the filtrate extracted with hexane, repeated the process three times. Dry the hexane fraction which contains ethyl esters. Take 2 mg of fish oil fatty acid ethyl ester and dissolved in 10 mll ethanol. Take 1 μl and 5 μl of the above fatty acid ethyl ester and add to 100 μl Streptococcus mutans solution (at final concentration of 1 μg/ml and 5 μg/ml), and culture overnight at 37° C. Dilute the overnight culture by 1:10000, take 10 μl of the above mixture and apply to blood agar plate, and cultured the plates overnight. Calculating the clone number on the plate, and compared with the negative control. Final concentration of 5 μg/ml algae extract could inhibit the growth of Streptococcus mutans. The study shows that fish oil fatty acid ethyl ester IC₅₀ is about 5 μg/ml.

### Example 6

The weight percentage composition of anti-microbial oral chewing tablet, containing fish oil fatty acids (EPA, or DHA, or ALA, or ARA or GLA)/or fish oil fatty acid methyl ester/or fish oil fatty acid ethyl ester, is as follows:

| | |
|---|---|
| Omega-3/omega-6 fatty acid/or fatty acid methyl ester/or | 1% |
| ethyl ester | |
| Mannitol/or xylitol or sorbitol | 5% |
| Dextrin | 18.5% |
| Menthol | 0.5% |
| HPMC | 4% |
| Manesium stearate | 0.5% |
| Other ingredients | 80% |

### Example 7

The weight percentage composition of chewing gum, containing fish oil fatty acids (EPA, DHA, ALA, ARA)/or fatty acid methyl ester/or fatty acid ethyl ester, is as follows:

| | |
|---|---|
| Omega-3/omega-6 fatty acids/or their methyl esters/or | 1-5% |
| ethyl esters | |
| Gum base | 80% |
| Mannitol, or xylitol or sorbitol | 10% |
| Menthol | 0.2% |
| Lecithin | 0.6% |
| Edible flavors (such as banana or other flavors) | 0.9% |
| Edible citric acid | 0.2% |

### Example 8

The weight percentage composition of chewing gum, containing fish oil fatty acids (EPA, or DHA, or ALA or ARA or GLA)/or fatty acid methyl esters/or fatty acid ethyl esters, is as follows:

| | |
|---|---|
| Omega-3/omega-6 fatty acids/or their methyl esters/or | 1-5% |
| ethyl esters | |
| Gum base | 80% |
| High intense sweetener mogroside, rebaudisoside | 2% |
| Menthol | 0.2% |
| Lecithin | 0.6% |
| Edible flavors (such as banana or other flavors) | 0.9% |
| Edible citric acid | 0.2% |

### Example 9

The weight percentage composition of dairy products, containing omega-3 fatty acids (EPA, or DHA, or ALA) or omega-6 fatty acid (ARA or GLA), or their fatty acid methyl ester/or fish oil fatty acid ethyl ester, is as follows:

| | |
|---|---|
| Omega-3 fatty acid/or methyl ester/or fish oil ethyl ester | 1-5% |
| Milk | 90-95% |
| Mannitol, or xylitol or sorbitol or syrup | 1-5% |
| Or high intense sweetener | 0.5~1% |

### Example 10

The weight percentage composition of candy, containing omega-3 fatty acids (EPA, DHA, ALA), or omega-6 fatty acid (ARA or GLA),/or omega fatty acid methyl ester/ethyl esters, is as follows:

| | |
|---|---|
| Fish oil/or fish oil methyl ester/or fish oil ethyl ester | 1-5% |
| Milk powder | 5% |
| Mannitol, or xylitol or sorbitol | 1% |
| Dry starch syrup solid objects | 30~35% |
| Non-fat milk solids | 5~10% |
| Plant fat | 15-20% |
| Salt | 0.2-0.3% |
| Colloidal | 1.5-2.0% |

### Example 11

The weight percentage composition of toothpaste, containing omega fatty acids (EPA, or DHA, or ALA, or ARA, or GLA)/or omega fatty acid methyl ester/or fish oil fatty acid ethyl ester, is as follows:

| | |
|---|---|
| Omega fatty acids/or omega fatty acid methyl ester/or ethyl | 1-5% |
| ester | |
| Baking soda | 35% |
| Xylitol | 5% |
| hydrogen peroxide | 5~10% |
| Plant fat | 15-20% |
| Flavors | 1.5-2.0% |

### Example 12

Take omega fatty acids (EPA, or DHA, or ALA, or ARA, GLA)/or their methyl ester/or ethyl ester dissolved in 20-50% ethanol aqueous solution, xylitol, sorbitol, or mogroside 1%, to make mouthwash. Conventional method is applied for preparation of the mouthwash production, where active content of extract is about 0.1-10%. The mouthwash can be used as mouth rinse for heat cough, sore throat, cavity, gum disease, periodontitis, for its antimicrobial activity.

### Example 13

The preparation of the health care toothpaste by weight of each component were made from fatty acids as the bioactive components, where active content of omega fatty acids (EPA, or DHA, or ALA, or ARA, GLA)/or their methyl ester/or ethyl ester dissolved is about 0.1-10%. The health care toothpaste can be used for against cavity, gum diseases, periodontal diseases, cough, sore throat or antimicrobial. Other ingredients include baking soda, glycerin, hydrogen peroxide and a trace amount of peppermint oil. The functional toothpaste can be used as mouth rinse for heat cough, sore throat, cavity, gum disease, periodontitis, for its antimicrobial activity.

### Example 14

The preparation of the functional candy by weight of each component were made as: Luo han kuo extract (1%), omega fatty acids (EPA, or DHA, or ALA, or ARA, GLA)/or their methyl ester/or ethyl ester dissolved is about 0.1-10%. The functional candy can be used to prevent cavity, gum disease, periodontitis with its antimicrobial activity.

### Example 15

The preparation of the functional ice cream was made by weight of each component were calculated as, where active content of extract is about 0.1-10%. The health ice cream can be used to prevent cavity, gum disease, periodontitis with its antimicrobial activity.

| | |
|---|---|
| Omega fatty acids, or esters | 0.01~5% |

½ cup milk or cream
1 tablespoon of sugar or 1/10 tablespoon of Lo Han Kuo extract
¼ teaspoon of vanilla extract or chocolate syrup
½ tablespoon rock salt or “cubed sodium chloride”
Transfer the whole mixture into “Ice Cream Maker” machine.

1. The present invention relates to the discovery of novel property of polyunsaturated fatty acids, including omega-3, omega-6, and omega-9 fatty acids, but not limited to; and their ester derivatives, and the discovery and application of the novel anti-microbial activity of omega-3, omega-6, and omega-9 fatty acids and their ester derivatives. Specifically, the omega-3, omega-6, omega-9 fatty acids, their fatty acid methyl esters, and fatty acid ethyl esters found in fish oil, algae oil, and plant oil, have anti-microbial activity against oral pathogens. The novel application of the fatty acids and their ester derivatives refers to the prevention and treatment of oral diseases.

2. As mentioned in claim 1, the application of the omega-3, omega-6, and omega-9 fatty acids and their ester derivatives for the prevention and treatment of oral diseases, include dental caries, periodontitis, gingivitis, toothache and bleeding, mouth ulcers, halitosis, pharyngitis, sore throat and so on. They are effective against many oral pathogens, for example, Streptococcus mutans (ATCC 25175), Porphyromonas gingivalis ATCC 33277, A. actinomycecomitan JP2, F. nucleatum ATCC 25586, and yeast Candida albicans ATCC 2091, but not limited to.

3. As mentioned in claim 1, the application of omega-3, omega-6, and omega-9 fatty acids; omega-3, omega-6, and omega-9 fatty acid methyl esters; and omega-3, omega-6, and omega-9 ethyl esters, derived from fish oil or algae oil or plant oil, in the prevention and treatment of oral diseases, the characteristics of the fatty acids are the extracts from fish oil or algae oil or plant oil, such as the common omega-3 polyunsaturated fatty acids, eicosapentaenoic acid (EPA), docosahexaenoic Acid (DHA), α-linolenic acid (ALA); omega-6 polyunsaturated fatty acids, arachidonic acid (ARA) and γ-linolenic acid (GLA); omega-9 polyunsaturated fatty acid, eicosenoic acid; and other common or unusual fatty acids in the sum, as well as their fatty acid methyl esters and fatty acid ethyl esters; but not limited to.

4. As mentioned in claim 1, the application of omega-3, omega-6, and omega-9 fatty acids; omega-3, omega-6, and omega-9 fatty acid methyl esters; and omega-3, omega-6, and omega-9 fatty acid ethyl esters found in fish oil, algae oil or plant oil in the prevention and treatment of oral diseases, the characteristic of health care include medicines, dietary supplements, food and daily necessities. Formulations of these products include omega-3, omega-6, and omega-9 fatty acids, omega-3, omega-6, and omega-9 fatty acid methyl esters, and omega-3, omega-6, and omega-9 fatty acid ethyl esters, additives and the appropriate form of the product carrier, and in accordance with these products prepared by conventional production methods. The omega fatty acids, and their ester derivatives are applied for prevention and treatment of dental caries, periodontitis, gingivitis, toothache and bleeding, mouth ulcers, halitosis, pharyngitis, and sore throat, oral bacteria-induced inflammation, canker sores and oral lesions, abscesses.

5. As mentioned in claim 4, the role of oral treatment with these drugs can be used in any form of formulations, include tablets, sprays, powder, gum, film, paint, ointment, gel, paste, liquid, foam, gel, mouthwash, which mainly refer to the tablets for oral medication in any form of tablets, including oral tablets, bio-adhesive patches, dispersible tablets, effervescent tablets, chewable/chewing tablets, chewable/chewing gum, tablets and other solutions. The omega-3, omega-6, and omega-9 fatty acids, omega-3, omega-6, and omega-9 fatty acid methyl esters, and omega-3, omega-6, and omega-9 fatty acid ethyl esters in the above-mentioned drugs at least for the content of 0.01 percent to the weight of the mixture of drugs, generally 0.01-20% better, but not limited to.

6. As mentioned in claim 4, the key role with the oral food include chewing gum, beverages, coffee, and other dairy products, the above-mentioned omega-3, omega-6, and omega-9 fatty acids, their fatty acid methyl esters, and their fatty acid ethyl esters in the above-mentioned content of food for at least 0.01% to weight mixture of food, generally 0.01-20% better, but not limited to.

7. As mentioned in claim 4, the key role with the oral daily necessities, include toothpaste, tooth powder, mouthwash; the above-mentioned omega-3, omega-6, and omega-9 fatty acids, their fatty acid methyl esters and ethyl esters in the daily necessities for the content of at least 0.01% to the weight of a mixture of daily necessities, generally 0.01-20% better, but not limited to.
